(19) United States
(12) Patent Application Publication
(10) Pub. No.: US 2026/0215765 A1
(43) Pub. Date: Jul. 30, 2026

(54) MUCOSAL CELL COLLECTION DEVICE AND MUCOSAL CELL COLLECTION METHOD

(71) Applicant: TOHOKU UNIVERSITY, Sendai-shi, Miyagi (JP)

(72) Inventors: Yoichi HAGA, Sendai-shi (JP); Noriko TSURUOKA, Sendai-shi (JP); Yang YE, Sendai-shi (JP); Soki OJIMA, Sendai-shi (JP)

(21) Appl. No.: 19/148,934

(22) PCT Filed: Jan. 18, 2024
(86) PCT No.: PCT/JP2024/001274

§ 371 Date: Jul. 17, 2025

(30) Foreign Application Priority Data
Jan. 23, 2023 (JP) 2023-008199

(51) Int. Cl. A61B 10/02; A61B 17/32

(52) U.S. Cl. CPC A61B 10/02; A61B 17/320068; A61B 2017/320064; A61B 2217/007

(57) ABSTRACT

This mucosal cell collection device includes an ultrasonic irradiation means, a tubular member that is provided around the ultrasonic irradiation means and has a protruding portion protruding beyond one end of the ultrasonic irradiation means, an introduction means that introduces liquid into a space formed by the ultrasonic irradiation means, the protruding portion of the tubular member, and a subject, and an extraction means that extracts cells separated from the mucosa of the subject from the space together with the liquid.

### CROSS-REFERENCE TO RELATED APPLICATIONS

This application is the U.S. National Stage entry of International Application No. PCT/JP2024/001274, filed on Jan. 18, 2024, which, in turn, claims priority to JP Patent Application No. 2023-008199, filed on Jan. 23, 2023, both of which are hereby incorporated herein by reference in their entireties for all purposes Technical Field

The present invention relates to a mucosal cell collection device and a mucosal cell collection method.

### BACKGROUND ART

Regarding sleep disorders, survey results show that in Japan, about 21% of general adult population suffers from insomnia, and about 15% of general adult population is aware of daytime sleepiness. Sleep deprivation or sleep disorders that persist for a long period of time may increase susceptibility to lifestyle-related diseases and depression. It is important to deal with sleep disorders appropriately. Sleep disorders are thought to develop due to disruptions to a biological clock (circadian rhythm). Understanding the circadian rhythm is very useful in diagnosing and treating sleep disorders.

Expression levels of clock genes that control the circadian rhythm are thought to increase or decrease in accordance with a circadian rhythm cycle. By measuring the expression levels of clock genes in a sample and comparing them with a molecular timetable, it is possible to determine exact time of the biological clock corresponding to the time the sample has been collected. That is, in order to grasp the circadian rhythm of a person, it is necessary to collect cells every few hours during daily life and to measure the expression levels of clock genes. For this measurement, a device that can collect cells as needed during daily life is useful. In addition, it is expected that measuring the expression levels of proteins that are useful for diagnosis and evaluation of disease condition by collecting cells as needed leads to unprecedented testing and diagnoses.

The oral cavity is sometimes called a mirror that reflects an individual's health condition. Changes indicative of disease may appear as changes in the oral mucosa and may reveal systemic conditions such as diabetes or vitamin deficiencies, or local effects of chronic tobacco or alcohol intake.

Patent Document 1 describes a liquefaction collection device that collects cells from mucosal tissue without destroying cell membranes, in which a reservoir housing includes therein an ultrasonic generator and a liquefaction promoting medium LPM (such as PBS) that transmits ultrasonic energy to the mucosal tissue and converts tissue constituents into a dissolved state, the reservoir housing forms a closed space in which the ultrasonic generator, the LPM, and tissue are in contact, the LPM is introduced into the space from an injection system device through a tube, and a liquefied tissue sample is collected from the reservoir housing through a tube into a sample container by pressure of a suction pump that is continuously applied.

Patent Document 2 describes a probe, which is a tissue collection device of a diagnostic system that collects tumor markers in prostate tissue, that includes a tubular cartridge that comes into contact with the tissue, and that, within a chamber that is an internal space of the cartridge, includes an ultrasonic transducer, an aluminum rod that transmits energy of the ultrasonic transducer, and liquid vibration coupling medium that fills a space between a tip of the rod and the tissue, and that the medium generates cavitation to effectively transmit ultrasonic energy to the tissue to collect epithelial cells.

Patent Document 3 describes a system for collecting Helicobacter pylori, in which liquid is injected from outside the body into the stomach through an injection channel formed in an insertion portion of an endoscope that is inserted into the human body, an ultrasonic transducer introduced into the stomach through an inner cavity of the endoscope applies ultrasonic vibrations to an inner wall of the stomach through the liquid injected into the stomach, an aspirator aspirates the liquid in the stomach, and the Helicobacter pylori is collected by filtering with a bacteria collection filter on an aspiration line.

Non-Patent Document 1 describes a portable surfactant-based tissue acquisition for a molecular profiling (STAMP) device that has one side of an ultrasonic element and a sampling buffer solution in an internal space of a tubular housing that comes into contact with tissue, and collects nucleic acids, proteins, and the like in the same ratio as in vivo.

### CITATION LIST

### Patent Document

Patent Document 1: JP 2012-518171 T
Patent Document 2: U.S. Pat. No. 6,589,173
Patent Document 3: JP 2001-245890 A

### Non-Patent Document

Non-Patent Document 1: Sumit Paliwal and two others, “One-step acquisition of functional biomolecules from tissues”, Proceedings of the National Academy of Sciences, Aug. 17, 2010, Vol. 107, No. 33, pp. 14627-14632

### SUMMARY OF INVENTION

### Technical Problem

Cells are commonly collected from blood or oral mucosa. Collecting cells from blood requires inserting a needle into a blood vessel, which is highly invasive, and may make it difficult to collect cells multiple times a day. Collecting cells from oral mucosa also requires scraping cells from the oral mucosa, which may destroy the cells and interfere with analysis, or cause the cells to be contaminated by substances in the oral cavity, which may also interfere with analysis.

The present invention has been made in view of the above circumstances, and an object of the present invention is to provide a minimally invasive mucosal cell collection device and mucosal cell collection method.

### Solution to Problem

The inventors have conducted intensive studies to achieve the above object and found that mucosal cells can be collected in a minimally invasive manner by irradiating the mucosa of a subject with ultrasound through liquid and extracting the cells separated from the mucosa of the subject together with the liquid, thereby completing the present invention.

The present invention includes the following aspects.

[1]A mucosal cell collection device including an ultrasonic irradiation means, a tubular member provided around the ultrasonic irradiation means and having a protruding portion protruding beyond one end of the ultrasonic irradiation means, an introduction means configured to introduce liquid into a space formed by the ultrasonic irradiation means, the protruding portion of the tubular member, and a subject, and an extraction means configured to extract a cell separated from mucosa of the subject from the space together with the liquid.

[2] The mucosal cell collection device according to [1], in which the ultrasonic irradiation means includes an ultrasonic transducer and an ultrasonic transmission member configured to transmit ultrasound generated by the ultrasonic transducer.

[3] The mucosal cell collection device according to [1] or [2], in which the introduction means and the extraction means each include a tube.

[4] The mucosal cell collection device according to [1] or [2], including a sealing member between the ultrasonic irradiation means and the tubular member.

[5] The mucosal cell collection device according to [2], in which the ultrasonic transducer includes a disc portion formed in a disc shape and is configured to be capable of vibrating in a lateral direction along a radial direction of the disc portion.

[6] The mucosal cell collection device according to [5], in which the ultrasonic transmission member converts vibration of the ultrasonic transducer into vibration in a longitudinal direction orthogonal to the lateral direction.

[7] The mucosal cell collection device according to [6], in which the ultrasonic transmission member is formed in a cymbal shape.

[8] The mucosal cell collection device according to [7], in which the ultrasonic transmission member includes a peripheral portion connected to the disc portion, an inclined portion connected to the peripheral portion and extending at an angle away from the disc portion, and a protruding portion connected to the inclined portion and protruding in a direction opposite to the disc portion.

[9] The mucosal cell collection device according to [8], in which the tubular member is provided around the protruding portion.

[10] The mucosal cell collection device according to [9], in which a maximum size in the lateral direction is 11 mm or less, and a maximum size in the longitudinal direction is 10 mm or less.

[11] The mucosal cell collection device according to [2], in which the ultrasonic transmission member is formed in a shape in which a cross-sectional area orthogonal to an axial direction of the ultrasonic transmission member decreases toward a tip of the protruding portion.

[12] The mucosal cell collection device according to [11], in which the tubular member is formed in a shape in which a cross-sectional area orthogonal to an axial direction of the tubular member decreases toward the tip of the protruding portion.

[13] The mucosal cell collection device according to [2], in which the tubular member is attached to the ultrasonic transmission member at a position corresponding to a node when the ultrasonic transmission member vibrates due to ultrasound generated by the ultrasonic transducer.

[14] The mucosal cell collection device according to [13], including a sealing member between the ultrasonic transmission member and the tubular member, in which the sealing member is attached to the ultrasonic transmission member and/or the tubular member at the position corresponding to the node.

[15]A method for collecting a mucosal cell using a mucosal cell collection device, the mucosal cell collection device including an ultrasonic irradiation means, a tubular member provided around the ultrasonic irradiation means and having a protruding portion protruding beyond one end of the ultrasonic irradiation means, an introduction means configured to introduce liquid into a space formed by the ultrasonic irradiation means, the protruding portion of the tubular member, and a subject, and an extraction means configured to extract a cell separated from mucosa of the subject from the space together with the liquid, the method including irradiating the mucosa of the subject with ultrasound through the liquid, and extracting the cell separated from the mucosa of the subject together with the liquid.

[16] The method for collecting a mucosal cell according to claim 15, further including washing away a substance in an oral cavity in the space and/or a substance in the oral cavity adhering to a surface of the mucosa of the subject with the liquid by filling the space formed by the ultrasonic irradiation means, the protruding portion of the tubular member, and the subject with the liquid and circulating the liquid prior to the irradiating or at an early stage of the irradiating.

### Advantageous Effects of Invention

According to the present invention, a minimally invasive mucosal cell collection device and mucosal cell collection method can be provided.

Further, according to the present invention, scraping cells from the oral mucosa is not required, and the cells are less likely to be destroyed, thus not interfering with analysis. Furthermore, according to the present invention, a mucosal surface is washed before cell collection, so substances in the oral cavity that can interfere with analysis are less likely to contaminate the collected mucosal cells, thus not interfering with analysis.

### BRIEF DESCRIPTION OF DRAWINGS

FIG. 1 is a longitudinal cross-sectional view of a mucosal cell collection device according to an embodiment of the present invention.

FIG. 2 is an end view of the mucosal cell collection device in FIG. 1 viewed from a lower end thereof.

FIG. 3 is a diagram illustrating a mucosal cell collection method according to an embodiment of the present invention.

FIG. 4 is a graph showing measurement results in Experimental Example 1. In the graph, the horizontal axis is frequency [kHz], and the vertical axis is ultrasonic intensity [W/cm²].

FIG. 5 is a graph showing measurement results in Experimental Example 2. In the graph, the horizontal axis is applied voltage [Vp-p], and the vertical axis is ultrasonic intensity [W/cm²].

FIG. 6 is photographs showing an overview of an experimental procedure in Experimental Example 3.

FIG. 7 is a diagram illustrating an experiment schedule in Experimental Example 3.

FIG. 8 is photographs (fluorescence microscope, eyepiece) showing results in Experimental Example 3.

FIG. 9 is photographs (fluorescence microscope, scan) showing results in Experimental Example 3.

FIG. 10 is a diagram illustrating an experiment schedule in Experimental Example 4.

FIG. 11 is a diagram illustrating an experiment schedule in Experimental Example 4.

FIG. 12 is a diagram illustrating an experiment schedule in Experimental Example 5.

FIG. 13 is a graph showing results in Experimental Example 4. In the graph, the horizontal axis is applied voltage [Vp-p], and the vertical axis is the number of cells collected.

FIG. 14 is a diagram illustrating an experiment schedule in Experimental Example 6.

FIG. 15 is a diagram illustrating an experiment schedule in Experimental Example 6.

FIG. 16 is a diagram illustrating an experiment schedule in Experimental Example 6.

FIG. 17 is a graph showing results in Experimental Example 6. In the graph, the horizontal axis is collection time [min], and the vertical axis is the number of cells collected.

FIG. 18 is a diagram illustrating an experiment schedule in Experimental Example 7.

FIG. 19 is a diagram illustrating an experiment schedule in Experimental Example 7.

FIG. 20 is a graph showing results in Experimental Example 7. In the graph, the horizontal axis is immersion time [min], and the vertical axis is the number of cells collected.

FIG. 21 is a diagram illustrating an experiment schedule in Experimental Example 8.

FIG. 22 is a graph showing results in Experimental Example 8. In the graph, the horizontal axis is the number of times of collection, and the vertical axis is the number of cells collected.

FIG. 23 is a perspective view of a mucosal cell collection device according to a second embodiment.

FIG. 24 is a perspective view illustrating an example in which an endoscope is applied to the mucosal cell collection device according to the second embodiment.

FIG. 25 is a longitudinal cross-sectional view of the mucosal cell collection device according to the second embodiment.

FIG. 26 is a diagram for describing conversion of a vibration direction by an ultrasonic transmission member according to the second embodiment.

FIG. 27 is a longitudinal cross-sectional view of a mucosal cell collection device according to a third embodiment.

FIG. 28 is an end view of the mucosal cell collection device in FIG. 27 viewed from a lower end thereof.

FIG. 29 is a diagram for describing an attachment position of a tubular member and/or a sealing member according to the third embodiment.

FIG. 30 is a diagram illustrating positions (1) to (4) as attachment positions of the sealing member.

FIG. 31 is a diagram showing a relationship between the attachment position of the sealing member and a relative value of output ultrasonic intensity.

### DESCRIPTION OF EMBODIMENTS

In this specification and claims, “to” indicating a range of numerical value means that numerical values described before and after “to” are included as a lower limit and an upper limit.

Hereinafter, embodiments of the present invention will be described with reference to the drawings as appropriate. However, the present invention is not limited to the embodiments and the drawings described below, and various modifications are possible without departing from the gist of the present invention.

### Mucosal Cell Collection Device

A mucosal cell collection device of the present invention includes an ultrasonic irradiation means, a tubular member that is provided around the ultrasonic irradiation means and has a protruding portion protruding beyond one end of the ultrasonic irradiation means, an introduction means that introduces liquid into a space formed by the ultrasonic irradiation means, the protruding portion of the tubular member, and a subject, and an extraction means that extracts cells separated from the mucosa of the subject from the space together with the liquid.

The mucosal cell collection device of the present invention will be described below with reference to an embodiment.

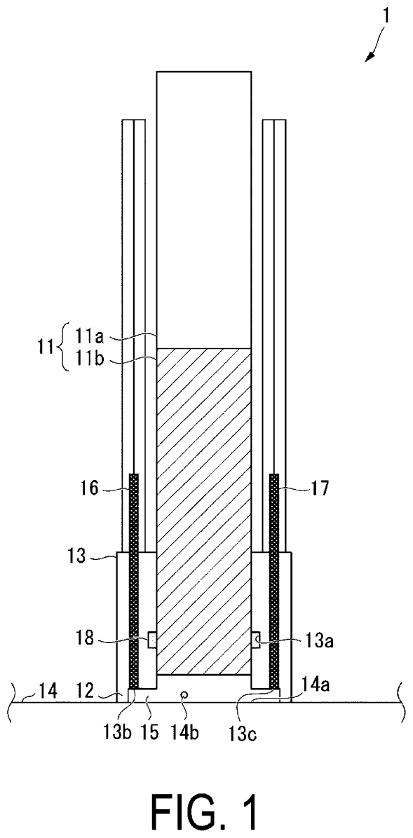

A mucosal cell collection device 1 illustrated in longitudinal section in FIG. 1 includes an ultrasonic irradiation means 11, a tubular member 13 that is provided around the ultrasonic irradiation means 11 and has a protruding portion 12 protruding beyond one end of the ultrasonic irradiation means 11, an introduction means 16 that introduces liquid into a space 15 formed by the ultrasonic irradiation means 11, the protruding portion 12 of the tubular member 13, and a subject 14, and an extraction means 17 that extracts cells separated from the mucosa 14a of the subject 14 together with the liquid from the space 15.

The ultrasonic irradiation means 11 includes, for example, an ultrasonic transducer 11a and an ultrasonic transmission member 11b that transmits ultrasound generated by the ultrasonic transducer 11a.

The ultrasonic transducer 11a is a member that converts high-frequency power from an oscillator into ultrasonic vibration. The ultrasonic transducer 11a is, for example, an electrostrictive type or a magnetostrictive type. The electrostrictive type expands and contracts when voltage is applied thereto, while the magnetostrictive type expands and contracts when a magnetic field is applied thereto, thereby generating ultrasound. As the ultrasonic transducer 11a, for example, a Langevin transducer, which is an electrostrictive ultrasonic transducer, is preferable. A resonance frequency of the ultrasonic transducer 11a is not limited, but is preferably 20 to 100 kHz.

The ultrasonic transmission member 11b is preferably made of a metal that has excellent transmission efficiency of ultrasound generated by the ultrasonic transducer 11a. Examples of metals include iron alloys such as steel, stainless steel, maraging steel, 42 alloy, Invar, Kovar, Sendust, Permendur, silicon steel, KS steel, and Spiegeleisen; alloy steels such as Krupp steel, chromium molybdenum steel, manganese molybdenum steel, and Yasugi steel; copper alloys such as brass, red brass, tombac, nickel silver, bronze, cupronickel, red copper, constantan, Nordic gold, and Knife; aluminum alloys such as duralumin, silumin, Hastelloy, Monel, Inconel, nichrome, sun platinum, and permalloy; and magnesium alloys.

A size of the ultrasonic irradiation means 11 can be set appropriately according to, for example, a site from which mucosal cells are to be collected. When used to collect oral mucosal cells, the size of the ultrasonic irradiation means 11 is preferably 0.05 to 0.2 m in length and 0.03 m or less in diameter when the ultrasonic irradiation means 11 is columnar.

A shape of the tubular member 13 is not limited as long as the shape is tubular, but a cylindrical shape is preferable. A material of the tubular member 13 is preferably a synthetic resin because the tubular member 13 is discarded each time cells are collected. The tubular member 13 can be manufactured by various methods such as injection molding and forming using a 3D printer.

The tubular member 13 is a separate member from the ultrasonic irradiation means 11. A sealing member is preferably included in an inner side surface of the tubular member 13 to prevent leakage of liquid from the space 15. An O-ring 18 is preferred as the sealing member, and a groove 13a into which the O-ring 18 is fitted is preferably provided on the inner side surface of the tubular member 13. The O-ring 18 is preferably elastically deformable, and can be made of, for example, natural rubber, synthetic rubber, or thermoplastic elastomer. The sealing member may be formed as an integral part of the tubular member 13.

A gap between the inner side surface of the tubular member 13 and the ultrasonic irradiation means 11 is not limited as long as the tubular member 13 can be attached to and detached from the ultrasonic irradiation means 11.

The tubular member 13 is provided with a through hole 13b that connects the space 15 to the introduction means 16, and a through hole 13c that connects the space 15 to the extraction means 17. When the tubular member 13 and at least part of the introduction means 16 and/or at least part of the extraction means 17 are formed as one piece, an inner cavity of the introduction means 16 and the through hole 13b and/or an inner cavity of the extraction means 17 and the through hole 13c are configured as one hole.

In the mucosal cell collection device 1, the tubular member 13 is preferably disposable, and the ultrasonic irradiation means 11 is preferably reusable. In particular, the tubular member 13 is preferably disposable because the tubular member 13 can be manufactured inexpensively and contamination can be avoided.

The introduction means 16 includes, for example, a tube. The introduction means 16 is connected to a reservoir (not illustrated) and a pump (not illustrated) for supplying liquid to be introduced into the space 15.

The extraction means 17 includes, for example, a tube. The extraction means 17 is connected to a collection tank (not illustrated) for collecting the liquid extracted from the space 15.

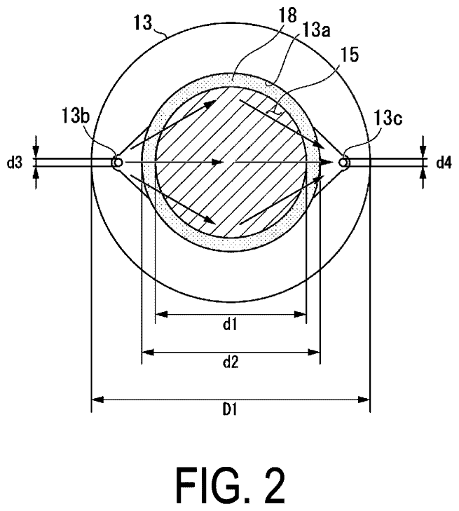

An end view illustrated in FIG. 2 is an end view viewed from a lower end of the mucosal cell collection device 1.

The tubular member 13, the through hole 13b, the through hole 13c, and the O-ring 18 are the same as those in FIG. 1. The groove 13a of the tubular member 13 cannot be viewed directly, but the O-ring 18 is fitted therein.

When the tubular member 13 has a cylindrical shape, the space 15 is a substantially columnar space, but slight protrusions are provided for introducing and extracting liquid through the through holes 13b and 13c.

When the tubular member 13 has a cylindrical shape, an outer diameter D1 thereof, a diameter d1 of the substantially columnar space 15, and a diameter d2 (=d1+a depth of the groove 13a×2) of a portion including the groove 13a are not limited. In addition, a diameter d3 of the through hole 13b and a diameter d4 of the through hole 13c are not limited.

The subject 14 is preferably a mammalian-related subject, such as a human.

The mucosa 14a is tissue that is always moistened with mucus, and is located on inner walls of the digestive tract, respiratory organs, excretory organ, reproductive organs, and the like. The mucosa 14a is an epithelial layer of ectoderm origin covered with epithelial cells and is involved in absorption and secretion. The mucosa 14a is located in various body cavities and faces external environment and internal organs. The mucosa 14a is connected to skin in various places, such as the nostrils, lips, ears, genitals, and anus. The mucosal epithelium is epithelium that protects a surface of the mucosa. Therefore, the mucosal epithelium is composed of “stratified squamous epithelium” in areas that are subjected to strong mechanical stimuli (such as the oral cavity, esophagus, and anus), and conversely, is composed of “simple columnar epithelium” in areas where secretion and absorption take place (such as the stomach and intestines). The lamina propria is a layer of connective tissue composed of dense collagen fibers. The muscularis mucosa is a thin layer of smooth muscle located below the lamina propria, which separates the lamina propria from the submucosa.

Examples of the mucosa 14a include buccal mucosa, gastric mucosa, intestinal mucosa, olfactory epithelium, oral mucosa, and endometrium. Since cells can be easily collected using the mucosal cell collection device in the present embodiment, the oral mucosa and the buccal mucosa are preferred. The gastric mucosa, the intestinal mucosa, the endometrium, and the like are also preferred when the mucosal cell collection device in the present embodiment can be brought into contact with the mucosa via a forceps port of the endoscope. The endometrium is mucosal tissue that covers the inside of the uterus. The mucosal cell collection device in the present embodiment can be applied to collect cells from serous membranes on surfaces of organs in the abdominal cavity, the thoracic cavity, and the like, as well as from the pia mater in the ventricles, in addition to the mucosa. The serous membrane is a membrane that covers surfaces of body cavities and organs within the body cavities and secretes serous fluid. The pia mater is the innermost of the meninges that surround the brain and spinal cord, and is a thin reticular membrane that completely covers a surface of the brain, including the inner surface of the ventricles.

The aforementioned liquid may be a buffer solution that causes little damage to cells, such as phosphate-buffered saline (PBS).

### Mucosal Cell Collection Method

A mucosal cell collection method in the present embodiment includes a step of irradiating the mucosa of a subject with ultrasound through liquid, and a step of extracting cells separated from the mucosa of the subject together with the liquid.

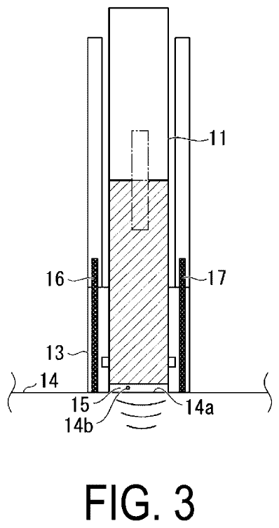

In the mucosal cell collection method in the present embodiment, as illustrated in FIG. 3, the mucosal cell collection device 1 is pressed against the mucosa 14a of the subject 14, liquid is introduced into the space 15 through the introduction means 16, and the mucosa 14a of the subject 14 is irradiated with ultrasound generated by the ultrasonic irradiation means 11 through the liquid. Thus, cells 14b are separated from the mucosa 14a and dispersed in the liquid.

The mucosal cell collection method in the present embodiment may further include a step of washing away substances in the oral cavity in the space 15 and/or substances in the oral cavity adhering to the surface of the mucosa 14a of the subject 14 with the liquid by filling the space 15 formed by the ultrasonic irradiation means 11, the protruding portion 12 of the tubular member 13, and the subject 14 with the liquid and circulating the liquid prior to the step of irradiating or at an early stage of the step of irradiating.

Here, “early stage of the irradiation step” means the former part of the total step time of the irradiation step.

By including this step, cells with fewer impurities can be collected.

When the mucosal cell collection device 1 is pressed against the mucosa 14a of the subject 14, the space 15 may be made negative pressure so that the mucosal cell collection device 1 adheres to the mucosa 14a.

By flowing the liquid in the space 15 without applying ultrasound, substances that are present in the space 15 and that interfere with analysis can be washed away.

The liquid in which the cells 14b are dispersed can be extracted through the extraction means 17, whereby the cells can be collected from the mucosa.

In the mucosal cell collection device 1, the tubular member 13 is preferably disposable, and the ultrasonic irradiation means 11 is preferably reusable. In particular, the tubular member 13 is preferably disposable because the tubular member 13 can be manufactured inexpensively and contamination can be avoided.

### EXAMPLES

The present invention will be described in more detail with reference to examples, but the present invention is not to be understood as being limited to the examples described below.

### Experimental Example 1

Frequency characteristics of the mucosal cell collection device 1 illustrated in FIG. 1 were evaluated.

A Langevin transducer (HEC-1540P2BF, manufactured by HONDA ELECTRONICS Co., Ltd.; resonance frequency 40 kHz) was used as the ultrasonic transducer 11a, and a columnar metal material having a diameter of 14 mm made by cutting brass was used as the ultrasonic transmission member 11b. An outer diameter of the tubular member 13 was 21 mm, a diameter of the space 15 was 15 mm, and a diameter of the O-ring 18 was 17.1 mm.

The space 15 was filled with water so that the protruding portion 12 of the mucosal cell collection device 1 was immersed in the water. A hydrophone (TC4013-1) was placed at a position 1 mm from the lower end of the protruding portion 12, and a voltage of 20 Vp-p was applied to the ultrasonic transducer 11a to generate ultrasound.

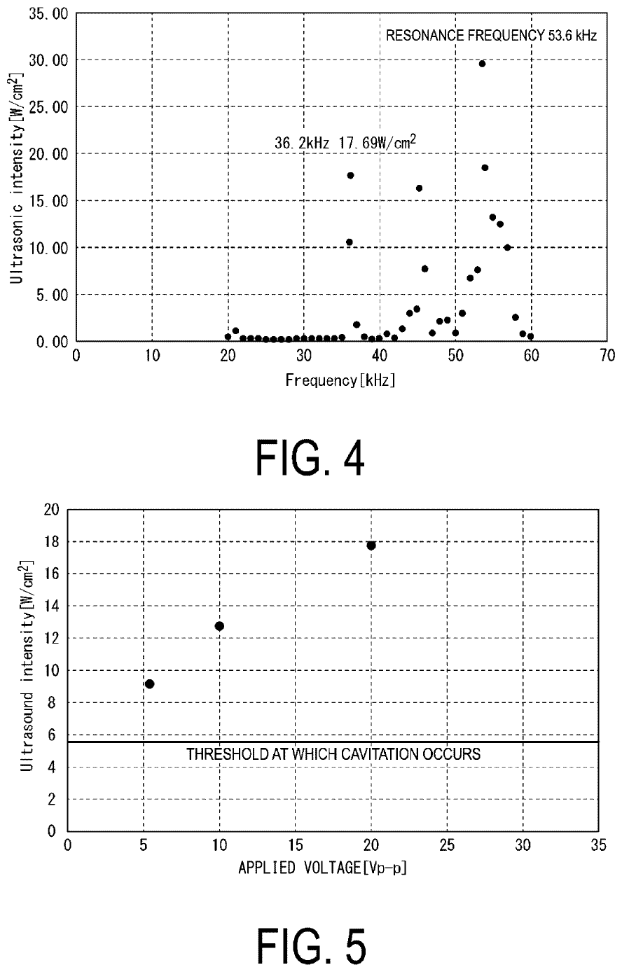

Measurement results are shown in FIG. 4. In a graph shown in FIG. 4. the horizontal axis is frequency [kHz] and the vertical axis is ultrasonic intensity [W/cm²].

### Experimental Example 2

With the same configuration as in Experimental Example 1, the ultrasonic intensity was measured by changing applied voltage.

Results are shown in FIG. 5. The frequency of the ultrasonic transducer 11a was 35.1 kHz. In a graph shown in FIG. 5, the horizontal axis is applied voltage [Vp-p], and the vertical axis is ultrasonic intensity [W/cm²].

### Experimental Example 3

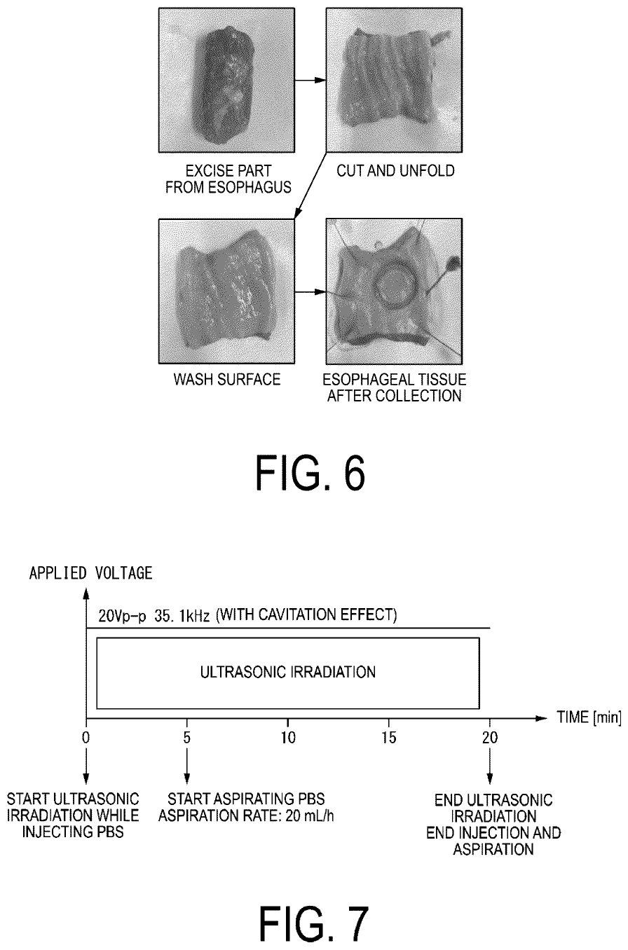

Part of the esophagus was excised from a pig (FIG. 6, upper left).

The excised esophagus was cut and unfolded (FIG. 6, upper right).

A surface was washed (FIG. 6, bottom left).

Using the same mucosal cell collection device as in Experimental Example 1, an experiment was conducted to collect cells from mucosal tissue excised from the esophagus of the pig. Phosphate-buffered saline (PBS) was used as the liquid for cell collection. The voltage applied to the ultrasonic transducer was 20 Vp-p and the frequency was 35.1 kHz. This is within a range in which a cavitation effect can be achieved.

As illustrated in FIG. 7, ultrasonic irradiation was started while injecting the PBS (0 min), aspiration of the PBS was started at an aspiration rate of 20 mL/h (5 min), and the ultrasonic irradiation and the injection and aspiration were finished (20 min).

The PBS containing the cells separated from the mucosa was collected and placed in a centrifuge tube, and the cells were precipitated by centrifugation.

The obtained cells were stained using a staining kit (Live/Dead Cell Staining Kit II, manufactured by PromoKine).

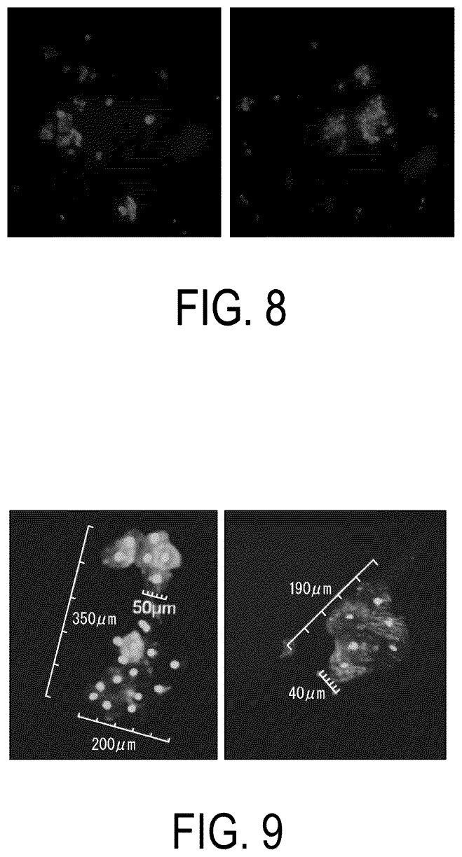

FIG. 8 shows results of observing the stained cells through an eyepiece using a fluorescence microscope (IX81, manufactured by Olympus Corporation) with excitation fluorescence at a wavelength of 488 nm, and FIG. 9 shows results of scanning from the fluorescence microscope.

The shape and size of the cells indicated that there was a high possibility that epithelial cells had been collected.

### Experimental Example 4

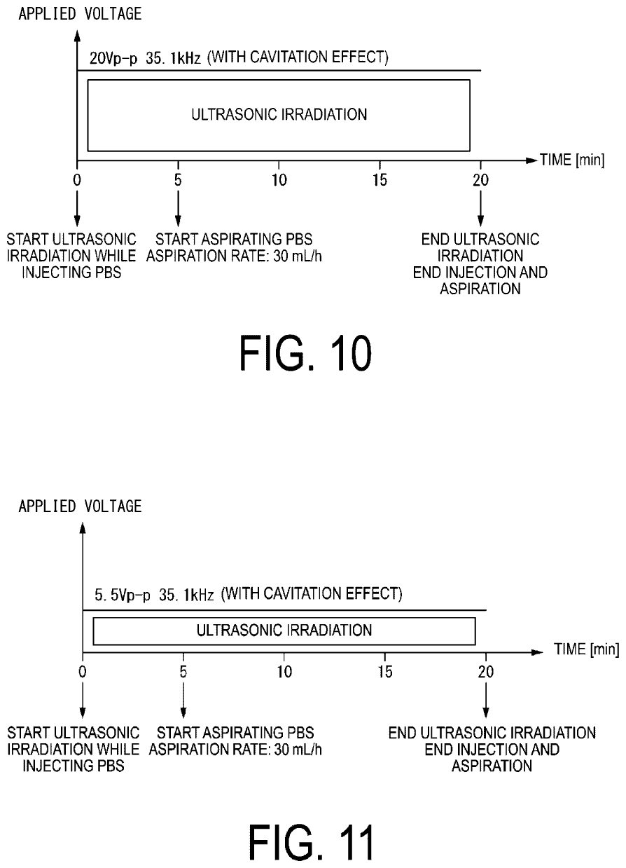

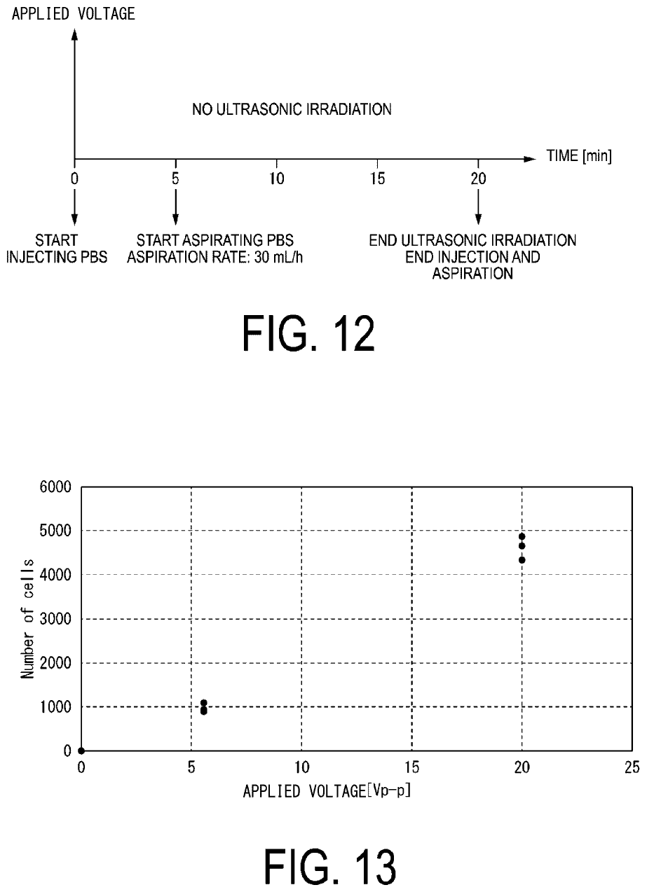

Experiments were conducted in the same manner as in Experimental Example 3, except that the ultrasonic intensity was varied as shown in FIGS. 10 to 12.

Results are shown in FIG. 13. In a graph in FIG. 13, the horizontal axis is applied voltage [Vp-p] and the vertical axis is the number of cells collected.

The higher the applied voltage (the stronger the ultrasonic intensity), the more cells were collected.

### Experimental Example 5 (Comparative Example)

An experiment was conducted in the same manner as in Experimental Example 3, except that the ultrasonic irradiation was not performed.

Only eight cells were collected.

### Experimental Example 6

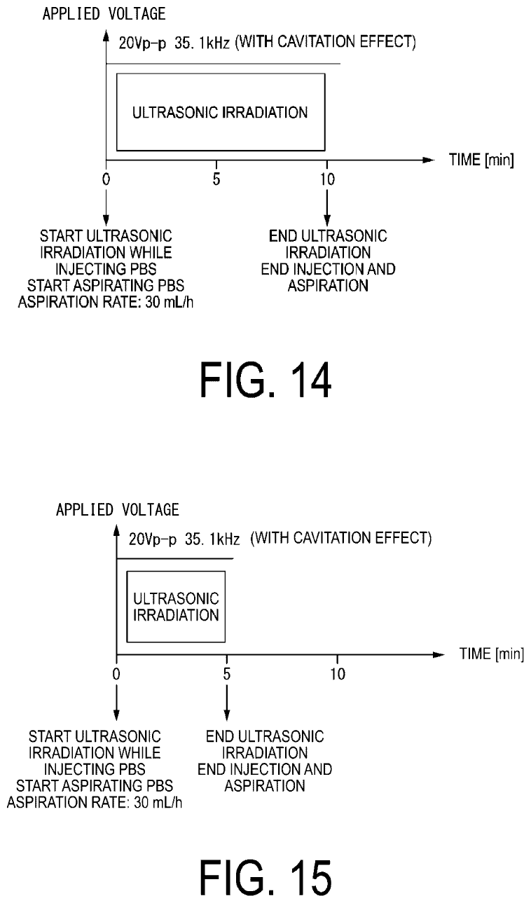

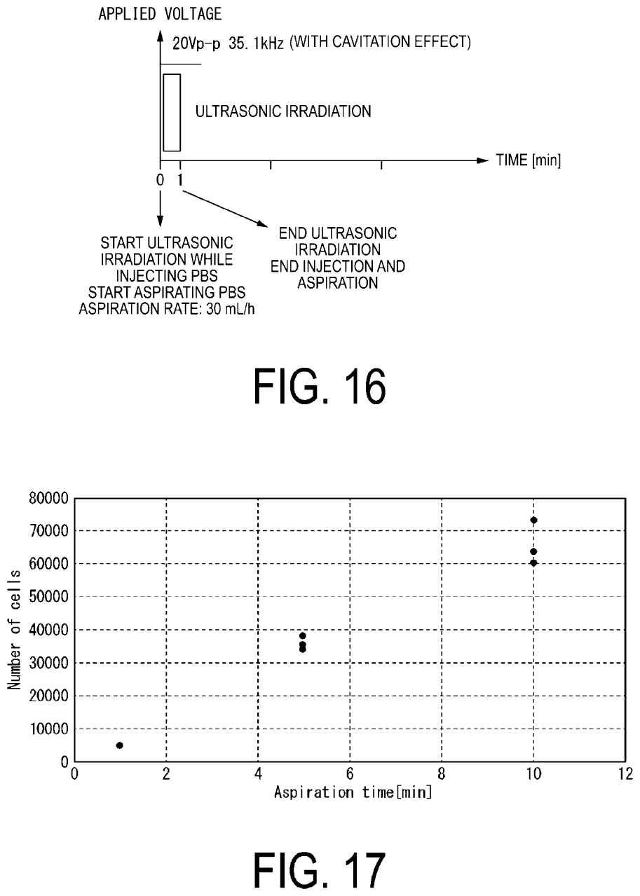

Experiments were conducted in the same manner as in Experimental Example 3, except that the cell collection time was varied as shown in FIGS. 14 to 16.

Results are shown in FIG. 17. In a graph in FIG. 17, the horizontal axis is collection time [min] and the vertical axis is the number of cells collected.

The longer the collection time, the more cells were collected.

### Experimental Example 7 (Comparative Example)

In order to confirm a difference in efficiency between the mucosal cell collection device and the common method of collecting cells from mucosal tissue using a swab, cell collection experiments were conducted by rubbing the mucosa with a swab.

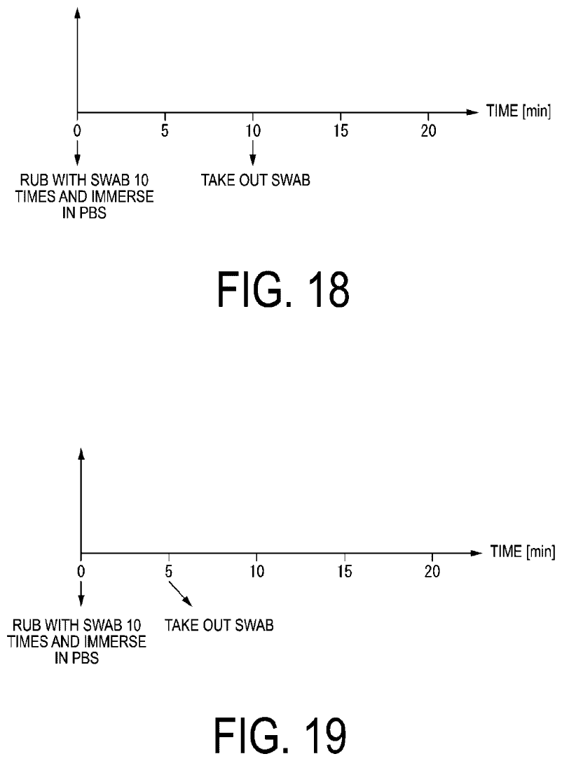

Experiment schedules are illustrated in FIGS. 18 and 19.

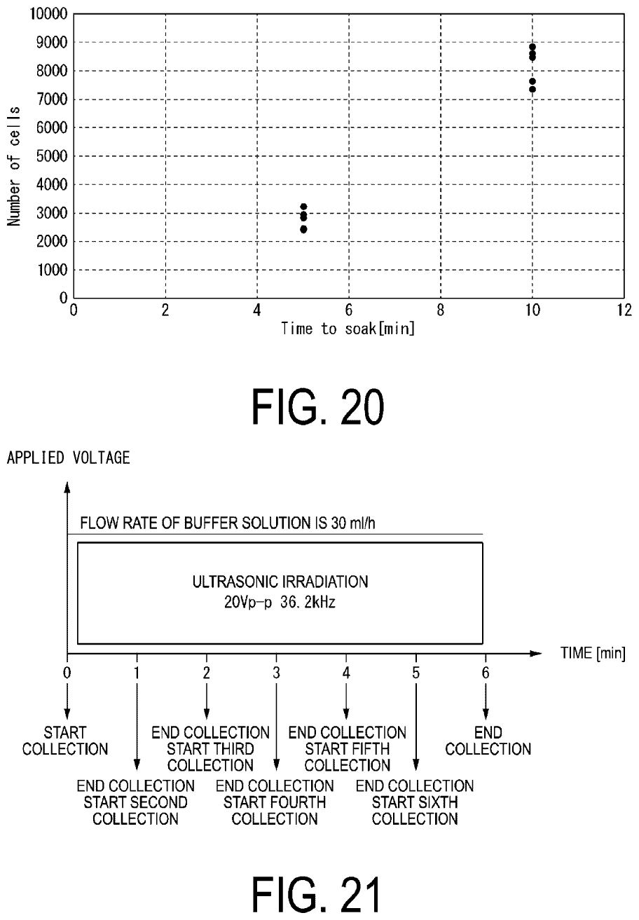

Results are shown in FIG. 20. In a graph in FIG. 20, the horizontal axis is immersion time [min] and the vertical axis is the number of cells collected.

The longer the immersion time, the more cells were collected, but the number of cells collected was smaller than when the mucosal cell collection device was used. Further, when the cells were collected with the swab, many of the cells were 14 μm or smaller in size, suggesting that impurities or fragmented cells were mixed in. Therefore, genetic analysis was expected to be difficult.

### Experimental Example 8

In order to measure dynamic changes in RNA transcription or protein expression from genes, cells are to be collected every four hours, that is, six times a day. The aim is to see what effect collecting cells from the same site six times has on the tissue.

An experiment was conducted according to an experiment schedule illustrated in FIG. 21.

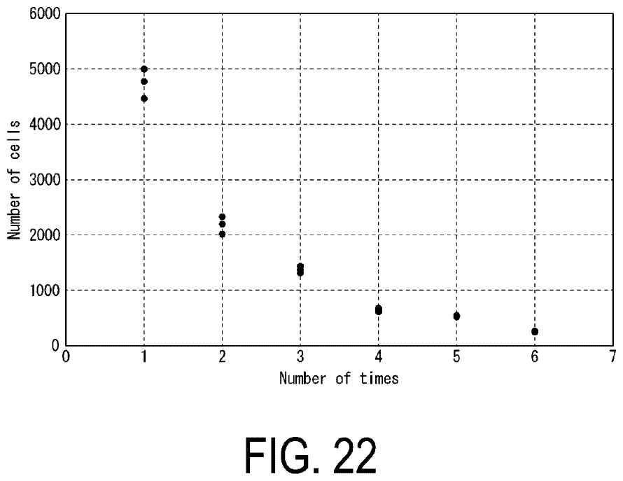

Results are shown in FIG. 22. In a graph in FIG. 22, the horizontal axis is the number of times of collection, and the vertical axis is the number of cells collected.

Approximately 3000 cells were collected in one-minute collection time at ultrasonic intensity slightly above the cavitation threshold.

It was also found that the mucosal cell collection device of the present invention can be used to collect cells multiple times in a day.

Next, a mucosal cell collection device 201 according to a second embodiment will be described.

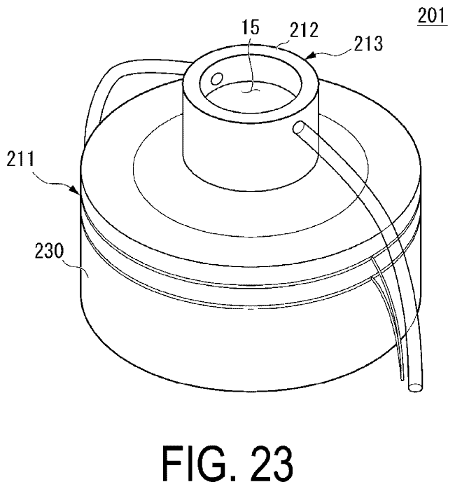

In the first embodiment, a Langevin transducer, which is an electrostrictive ultrasonic transducer, has been described as an example of the ultrasonic transducer 11a, but the ultrasonic transducer is not limited thereto. As illustrated in FIGS. 23 to 26, in the second embodiment, aspects of an ultrasonic transducer 220 and an ultrasonic transmission member 222 differ from those in the first embodiment described above. In the following description, the same reference numerals are given to the same components as those in the first embodiment described above, and detailed description thereof will be omitted.

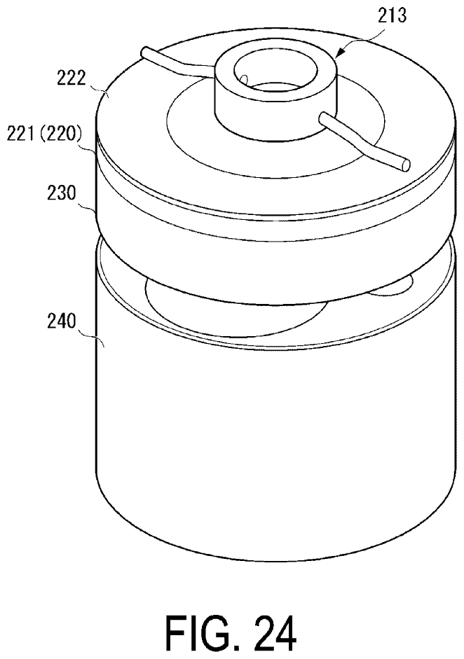

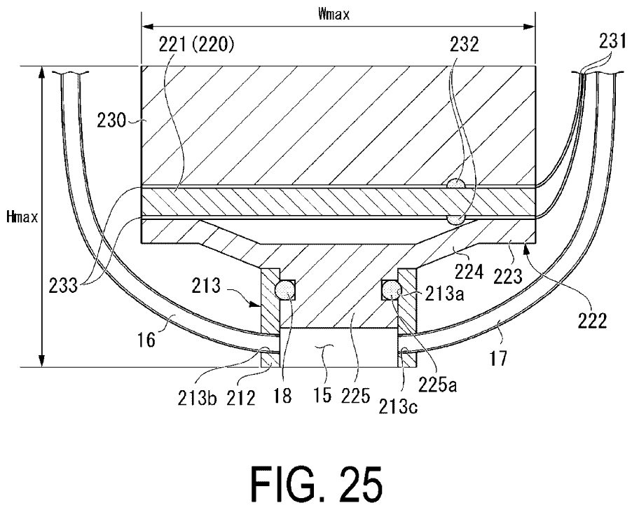

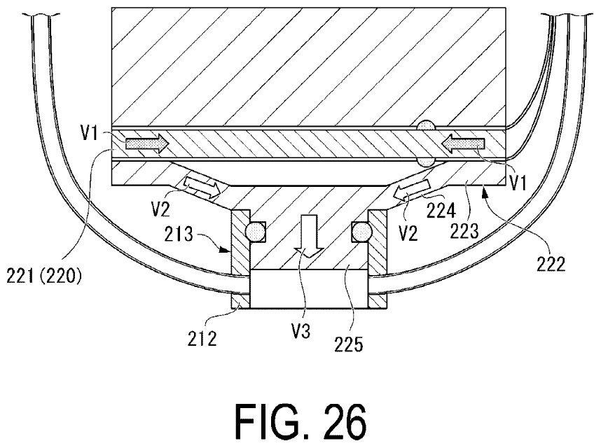

FIG. 23 is a perspective view of the mucosal cell collection device 201 according to the second embodiment. FIG. 24 is a perspective view illustrating an example in which an endoscope is applied to the mucosal cell collection device 201 according to the second embodiment. FIG. 25 is a longitudinal cross-sectional view of the mucosal cell collection device 201 according to the second embodiment. FIG. 26 is a diagram for describing conversion of a vibration direction by the ultrasonic transmission member 222 according to the second embodiment.

Referring to FIGS. 23 to 26 together, the mucosal cell collection device 201 includes an ultrasonic irradiation means 211, a support 230 that supports the ultrasonic irradiation means 211, a tubular member 213 that is provided around the ultrasonic irradiation means 211 and has a protruding portion 212 protruding beyond one end of the ultrasonic irradiation means 211, an introduction means 16 that introduces liquid into a space 15 formed by the ultrasonic irradiation means 211, the protruding portion 212 of the tubular member 213, and a subject 14, and an extraction means 17 that extracts cells separated from the mucosa 14a of the subject 14 from the space 15 together with the liquid.

The ultrasonic irradiation means 211 includes the ultrasonic transducer 220 and the ultrasonic transmission member 222 that transmits ultrasound generated by the ultrasonic transducer 220.

The ultrasonic transducer 220 includes a disc portion 221 formed in a disc shape. An example of the disc portion 221 is a piezoelectric element that converts force applied to a piezoelectric material into vibration. The piezoelectric element may be formed of, for example, lead zirconate titanate (PZT), which is a type of piezoelectric ceramic.

The ultrasonic transducer 220 is configured to be capable of vibrating in a lateral direction along a radial direction of the disc portion 221. Hereinafter, the vibration in the lateral direction is also referred to as “lateral vibration”. Electric wirings 231 are connected to an upper surface and a lower surface of the ultrasonic transducer 220. The electric wirings 231 may be connected to the ultrasonic transducer 220 using, for example, solder 232.

For example, when voltage is applied to the ultrasonic transducer 220 through the electric wirings 231, the ultrasonic transducer 220 vibrates (laterally vibrates) in a direction of arrow V1 in FIG. 26 (lateral vibration, radially inward in the illustrated example). The ultrasonic transmission member 222 converts the vibration of the ultrasonic transducer 220 into vibration in a longitudinal direction orthogonal to the lateral direction (hereinafter also referred to as “longitudinal vibration”). For example, when the ultrasonic transducer 220 vibrates in the direction of arrow V1 in FIG. 26 (lateral vibration, radially inward in the illustrated example), the ultrasonic transmission member 222 vibrates (longitudinally vibrates) in a direction of arrow V3 in FIG. 26 (longitudinal direction, downward in the illustrated example).

The ultrasonic transmission member 222 is preferably made of a metal that has excellent transmission efficiency of ultrasound generated by the ultrasonic transducer 220. Examples of metals include the same metals as those in the first embodiment described above.

The ultrasonic transmission member 222 is formed in a cymbal shape. The ultrasonic transmission member 222 includes a peripheral portion 223 connected to the disc portion 221, an inclined portion 224 connected to the peripheral portion 223 and extending at an angle away from the disc portion 221, and a protruding portion 225 connected to the inclined portion 224 and protruding in a direction opposite to the disc portion 221.

The peripheral portion 223 is formed in an annular shape along a periphery of the disc portion 221. The peripheral portion 223 may be connected to the periphery of a lower surface of the disc portion 221 using, for example, an adhesive 233 or the like.

The inclined portion 224 is formed in a conical shape (tapered shape) that inclines downward from an inner edge of the peripheral portion 223 toward the inside in a radial direction. For example, when the ultrasonic transducer 220 vibrates in the direction of arrow V1 in FIG. 26 (lateral vibration, radially inward in the illustrated example), the inclined portion 224 is displaced in a direction of arrow V2 in FIG. 26 (oblique direction, radially inward and downward in the illustrated example).

The protruding portion 225 is formed in a protruding shape that protrudes downward from an inner edge of the inclined portion 224. The protruding portion 225 is placed coaxially with the disc portion 221. An outer diameter of the protruding portion 225 is smaller than an outer diameter of the ultrasonic transducer 220.

The tubular member 213 is provided around the protruding portion 225 of the ultrasonic transmission member 222. A shape of the tubular member 213 is not limited as long as the shape is tubular, but is preferably cylindrical. The tubular member 213 is detachably attached to the protruding portion 225 of the ultrasonic transmission member 222. The tubular member 213 may be detachably attached to the protruding portion 225 using, for example, a screw structure. For example, a female thread (not illustrated) may be provided on an inner side surface of the tubular member 213, and a male thread (not illustrated) may be provided on an outer side surface of the protruding portion 225. The male thread of the protruding portion 225 may be screwed into the female thread of the tubular member 213 to enable attachment and detachment.

A sealing member is preferably included in the inner side surface of the tubular member 213 to prevent leakage of liquid from the space 15. An O-ring 18 is preferred as the sealing member. A groove 213a into which the O-ring 18 is fitted is preferably provided on the inner side surface of the tubular member 213. Note that the sealing member may be formed as an integral part of the tubular member 213. In this case, a groove into which the sealing member is fitted is preferably provided on the outer side surface of the protruding portion 225.

A groove 225a into which the O-ring 18 is fitted is preferably provided on the outer side surface of the protruding portion 225. Note that the sealing member may be formed as an integral part of the protruding portion 225. In this case, a groove into which the sealing member is fitted is preferably provided on the inner side surface of the tubular member 213.

The tubular member 213 is formed with a through hole 213b that connects the space 15 to the introduction means 16, and the through hole 213c that connects the space 15 to the extraction means 17. The through holes 213b and 213c open in the tubular member 213 in a radial direction of the tubular member 213. The through holes 213b and 213c are preferably located on opposite sides to each other in the radial direction of the tubular member 213.

The tubular member 213 and at least part of the introduction means 16 and/or at least part of the extraction means 17 may be formed as one piece. In this case, an inner cavity of the introduction means 16 and the through hole 213b and/or an inner cavity of the extraction means 17 and the through hole 213c are configured as one hole.

The introduction means 16 includes, for example, a tube. One end of the introduction means 16 is connected to the space 15. Another end of the introduction means 16 may be connected to a reservoir (not illustrated) and a pump (not illustrated) for supplying liquid to be introduced into the space 15.

The extraction means 17 includes, for example, a tube. One end of the extraction means 17 is connected to the space 15. Another end of the extraction means 17 may be connected to a collection tank (not illustrated) for collecting the liquid extracted from the space 15.

The support 230 is formed in a columnar shape. The support 230 is placed coaxially with the ultrasonic transducer 220. An outer diameter of the support 230 may be the same size as the outer diameter of the ultrasonic transducer 220. The support 230 may be connected to the upper surface of the ultrasonic transducer 220 using, for example, the adhesive 233 or the like. The support 230 serves to support the upper surface of the ultrasonic transducer 220 and prevent deflection of the ultrasonic transducer 220.

For example, a tip 240 of an endoscope (an example of an imaging device) may be attached to the support 230. The tip 240 of the endoscope is preferably detachably attached to a surface of the support 230 opposite to the ultrasonic transducer 220. In FIG. 24, outer diameter sizes of the tip 240 of the endoscope and the support 230 are approximately equal, but this is not necessarily the case. For example, the outer diameter sizes of the mucosal cell collection device 201 and the support 230 may be made smaller so as not to interfere with illumination and observation functions of the endoscope. The mucosal cell collection device 201 and the support 230 may be displaced from the center and positioned above an endoscope forceps port, for example. Further, a cylindrical resin cap may be attached to the tip 240 of the endoscope. In this case, the mucosal cell collection device 201 and the support 230 may be placed at a tip of the cap, and attached to the tip 240 of the endoscope with the cap therebetween.

A size of the mucosal cell collection device 201 can be set appropriately according to, for example, a site from which mucosal cells are to be collected. In the present embodiment, the mucosal cell collection device 201 has a maximum lateral dimension Wmax of 11 mm or less and a maximum longitudinal dimension Hmax of 10 mm or less. The maximum lateral dimension Wmax of the mucosal cell collection device 201 corresponds to a diameter of the ultrasonic transducer 220. The maximum longitudinal dimension Hmax of the mucosal cell collection device 201 corresponds to a distance between an upper end of the support 230 and a lower end of the protruding portion 212.

In the second embodiment described above, the ultrasonic transducer 220 includes the disc portion 221 formed in a disc shape. The ultrasonic transducer 220 is configured to be capable of vibrating in the lateral direction along the radial direction of the disc portion 221.

According to this configuration, the ultrasonic transducer 220 is more easily downsized in a thickness direction (longitudinal direction) compared to when the ultrasonic transducer is a Langevin transducer (when the ultrasonic transducer includes a block body). This contributes to making the mucosal cell collection device 201 smaller in the longitudinal direction and lighter in weight. For example, by making the mucosal cell collection device 201 smaller, cells can be more easily collected from the mucosa inside the body (e.g., inner walls of the digestive tract such as the stomach and intestines, and surfaces of abdominal organs).

In the second embodiment, the ultrasonic transmission member 222 converts the vibration of the ultrasonic transducer 220 into the vibration in the longitudinal direction orthogonal to the lateral direction.

According to this configuration, the lateral vibration of the ultrasonic transducer 220 can be converted into the longitudinal vibration, thereby producing the longitudinal vibration of the tip of the device (the lower end of the protruding portion 212).

In the second embodiment, the ultrasonic transmission member 222 is formed in a cymbal shape.

According to this configuration, the ultrasonic transmission member 222 is more easily downsized in the thickness direction (longitudinal direction) compared to when the ultrasonic transmission member is formed in a columnar shape. This contributes to making the mucosal cell collection device 201 smaller in the longitudinal direction and lighter in weight.

In the second embodiment, the ultrasonic transmission member 222 includes the peripheral portion 223 connected to the disc portion 221, the inclined portion 224 connected to the peripheral portion 223 and extending at an angle away from the disc portion 221, and the protruding portion 225 connected to the inclined portion 224 and protruding in the direction opposite to the disc portion 221.

According to this configuration, the lateral vibration of the disc portion 221 is converted into the longitudinal vibration of the protruding portion 225 by the mechanical bending deformation of the inclined portion 224 connected to the peripheral portion 223, thereby generating the longitudinal vibration at the tip of the device (the lower end of the protruding portion 212).

In the second embodiment, the tubular member 213 is provided around the protruding portion 225.

According to this configuration, the tubular member 213 can also be made smaller in correspondence with the protruding portion 225, which is highly beneficial when applied to specific applications (e.g., collection of samples from small animals and reduction of patient burden).

In the second embodiment, the mucosal cell collection device 201 has the maximum lateral dimension Wmax of 11 mm or less and the maximum longitudinal dimension Hmax of 10 mm or less.

This configuration contributes to making the mucosal cell collection device 201 smaller and lighter.

Next, a mucosal cell collection device 301 according to a third embodiment will be described.

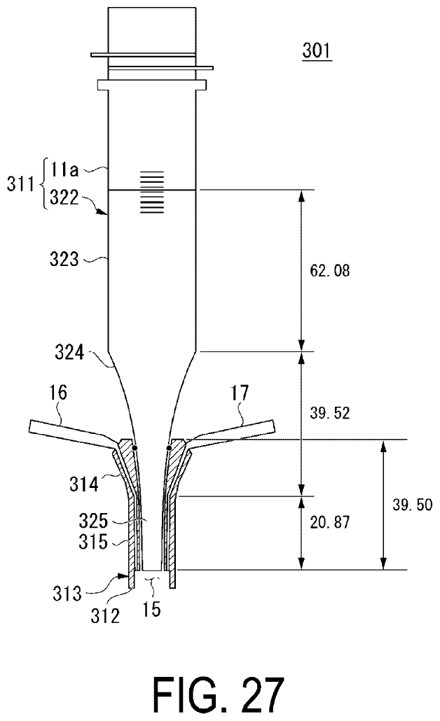

In the first embodiment, an example has been described in which the ultrasonic transmission member 11b is columnar and the tubular member 13 is cylindrical, but this is merely one example. As illustrated in FIGS. 27 to 29, in the third embodiment, aspects of an ultrasonic transmission member 322 and a tubular member 313 differ from those in the first embodiment described above. In the following description, the same reference numerals are given to the same components as those in the first embodiment described above, and detailed description thereof will be omitted.

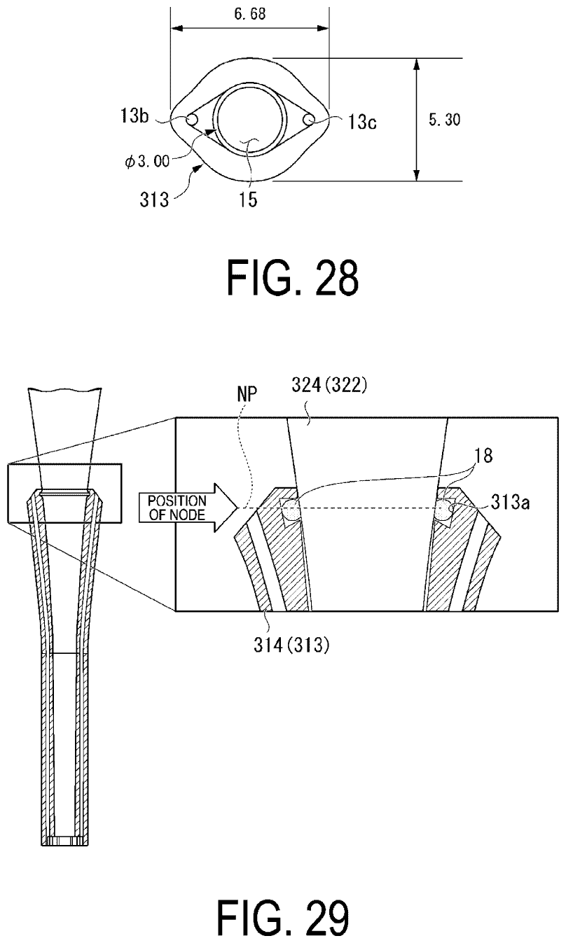

FIG. 27 is a longitudinal cross-sectional view of the mucosal cell collection device 301 according to the third embodiment. FIG. 28 is an end view of the mucosal cell collection device 301 in FIG. 27 viewed from a lower end. FIG. 29 is a diagram for describing an attachment position of the tubular member 313 and/or a sealing member according to the third embodiment. In FIGS. 27 and 29, the tubular member 313 of the mucosal cell collection device 301 is illustrated with a hatched cross section.

Referring to FIGS. 27 to 29 together, the mucosal cell collection device 301 includes an ultrasonic irradiation means 311, the tubular member 313 that is provided around the ultrasonic irradiation means 311 and has a protruding portion 312 protruding beyond one end of the ultrasonic irradiation means 311, an introduction means 16 that introduces liquid into a space 15 formed by the ultrasonic irradiation means 311, the protruding portion 312 of the tubular member 313, and a subject 14, and an extraction means 17 that extracts cells separated from the mucosa 14a of the subject 14 from the space 15 together with the liquid.

The ultrasonic irradiation means 311 includes an ultrasonic transducer 11a and the ultrasonic transmission member 322 that transmits ultrasound generated by the ultrasonic transducer 11a.

The ultrasonic transmission member 322 is formed in a shape in which a cross-sectional area orthogonal to an axial direction of the ultrasonic transmission member 322 decreases toward a tip of the protruding portion 312. The cross-sectional area orthogonal to the axial direction of the ultrasonic transmission member 322 corresponds to an area of the ultrasonic transmission member 322 in a lateral cross-sectional view (lateral cross-sectional area).

The ultrasonic transmission member 322 in the present embodiment corresponds to a horn. The horn is a transmission body in which a cross-sectional area in a longitudinal direction changes, unlike other transmission bodies, and has not only a function of transmitting vibration but also a function of transforming amplitude of vibration.

The ultrasonic transmission member 322 is formed, for example, in an exponential shape with straight portions connected to a large end face and a small end face of the exponential-shaped portion. In the present embodiment, the ultrasonic transmission member 322 includes a first straight portion 323 formed in a columnar shape extending in a straight line, a narrowing portion 324 connected to a lower end of the first straight portion 323 and narrowing in diameter toward a lower end thereof, and a second straight portion 325 connected to the lower end of the narrowing portion 324 and formed in a columnar shape extending in a straight line. In FIG. 27, as an example of dimensions of the ultrasonic transmission member 322, a longitudinal length of the first straight portion 323, a longitudinal length of the narrowing portion 324, and a longitudinal length of the second straight portion 325 (the upper reference line for the dimension 20.87 shown in FIG. 27 is a starting point of the straight portion of the horn and corresponds approximately to where the extraction means 17 bends) are shown (units [mm] are not shown), but the lengths of these portions are not limited to these.

Note that an aspect of the ultrasonic transmission member 322 is not limited to this. For example, the ultrasonic transmission member 322 may be formed in a stepped shape, a conical shape, a simple exponential shape, a catenoidal shape, a Fourier shape, a stepped composite shape, or the like.

When a Langevin transducer is included as the ultrasonic transducer 11a, the ultrasonic transmission member 322 is preferably shaped to resonate at the resonance frequency of the Langevin transducer. The ultrasonic transmission member 322 is preferably shaped to be easily inserted into the oral cavity. The ultrasonic transmission member 322 is preferably formed of a material with excellent fatigue strength, vibration characteristics, and corrosion resistance (e.g., a titanium alloy).

The tubular member 313 is formed in a shape in which a cross-sectional area orthogonal to an axial direction of the tubular member 313 decreases toward the tip of the protruding portion 312. The cross-sectional area orthogonal to the axial direction of the tubular member 313 corresponds to an area of the tubular member 313 in a lateral cross-sectional view (lateral cross-sectional area).

The tubular member 313 is shaped, for example, along an outer shape of part of the ultrasonic transmission member 322. In the present embodiment, the tubular member 313 includes a tapered portion 314 that extends along a lower part of the narrowing portion 324 of the ultrasonic transmission member 322 and narrows in diameter toward a lower end thereof, and a straight tube portion 315 that extends along the second straight portion 325 of the ultrasonic transmission member 322, is connected to the lower end of the tapered portion 314, and is formed in a cylindrical shape so as to extend in a straight line. Note that an aspect of the tubular member 313 is not limited to this. In FIG. 27, as an example of a dimension of the tubular member 313, a longitudinal length of a portion of the tubular member 313 excluding the protruding portion 312 is shown (unit [mm] is not shown), but the dimension is not limited to this. In FIG. 28, as an example of dimensions of the tubular member 313, lengths of the tubular member 313 in a major axis direction and a minor axis direction when viewed from the lower end of the mucosal cell collection device 301, and an inner diameter of the straight tube portion 315 (corresponding to a diameter d1 of the approximately columnar space 15 described above) are shown (units [mm] are not shown), but the dimensions are not limited to these.

The tubular member 313 is attached to the ultrasonic transmission member 322 at a position NP that corresponds to a node when the ultrasonic transmission member 322 vibrates due to ultrasound generated by the ultrasonic transducer 11a. The position NP that corresponds to the node when the ultrasonic transmission member 322 vibrates corresponds to a position that does not vibrate when a standing wave is applied to the ultrasonic transmission member 322 (a position different from a position that vibrates and corresponds to an antinode).

The mucosal cell collection device 301 includes an O-ring 18 (an example of a sealing member) between the ultrasonic transmission member 322 and the tubular member 313. The O-ring 18 is attached to the ultrasonic transmission member 322 and/or the tubular member 313 at the position NP, which corresponds to the node.

In the present embodiment, a groove 313a into which the O-ring 18 is fitted is provided on an inner side surface of the tubular member 313. The groove 313a is provided on an upper inner surface of the tapered portion 314 of the tubular member 313. Note that an aspect of the groove 313a is not limited to this.

In the present embodiment, the O-ring 18 is attached to the narrowing portion 324 of the ultrasonic transmission member 322 and the tapered portion 314 of the tubular member 313 at the position NP that corresponds to the node. Note that an aspect in which the O-ring 18 is attached is not limited to this.

In the third embodiment described above, the ultrasonic transmission member 322 is formed in a shape in which the cross-sectional area orthogonal to the axial direction of the ultrasonic transmission member 322 decreases toward the tip of the protruding portion 312.

This configuration is of large practical benefit when a collection portion at the tip is made thinner to suit specific applications (e.g., collection of samples from small animals and reduction of patient burden).

In the third embodiment, the tubular member 313 is formed in a shape in which the cross-sectional area orthogonal to the axial direction of the tubular member 313 decreases toward the tip of the protruding portion 312.

This configuration is of large practical benefit when a collection portion at the tip is made thinner to suit specific applications (e.g., collection of samples from small animals and reduction of patient burden).

In the third embodiment, the tubular member 313 is attached to the ultrasonic transmission member 322 at the position NP that corresponds to the node when the ultrasonic transmission member 322 vibrates due to ultrasound generated by the ultrasonic transducer 11a.

According to this configuration, the vibration of the ultrasonic transmission member 322 can be efficiently transmitted to the tubular member 313.

In the third embodiment, the O-ring 18 is provided between the ultrasonic transmission member 322 and the tubular member 313, and the O-ring 18 is attached to the ultrasonic transmission member 322 and the tubular member 313 at the position NP that corresponds to the node.

According to this configuration, even when the O-ring 18 is provided, the vibration of the ultrasonic transmission member 322 can be efficiently transmitted to the tubular member 313.

The present invention will be described in more detail below using an example of the attachment position of the sealing member, but the present invention is not to be understood as being limited to the example of the attachment position of the sealing member described below.

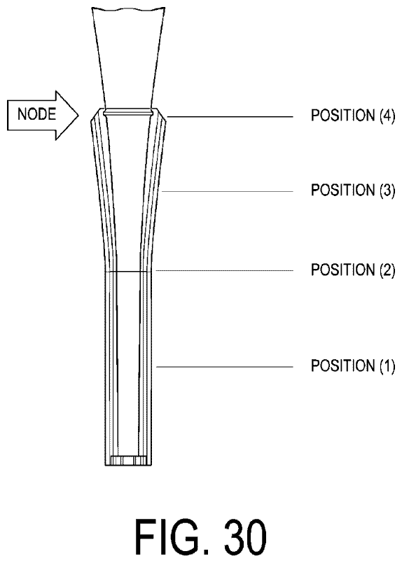

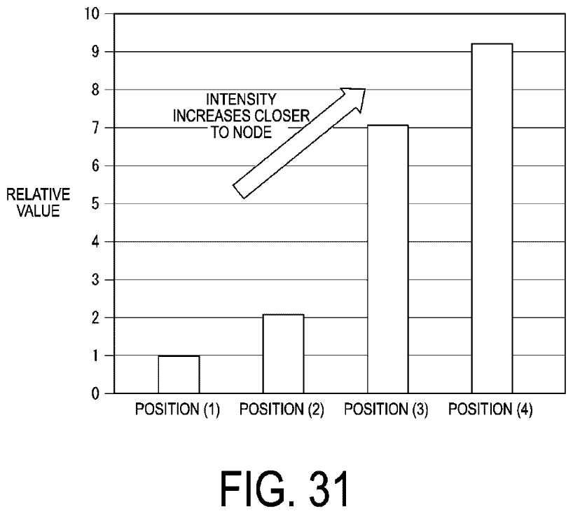

FIG. 30 is a diagram illustrating positions (1) to (4) as attachment positions of the sealing member. FIG. 31 is a diagram showing a relationship between the attachment position of the sealing member and the relative value of the output ultrasonic intensity.

As illustrated in FIG. 30, position (4) corresponds to the position corresponding to the node. Position (1) to position (4) are located in order near the position corresponding to the node.

The vertical axis in FIG. 31 shows the relative value when the intensity at position (1) is set to 1. As shown in FIG. 31, it was found that the intensity increased closer to the position corresponding to the node (position (4)).

### REFERENCE SIGNS LIST

1, 201, 301 Mucosal cell collection device
11, 211, 311 Ultrasonic irradiation means
11a, 220 Ultrasonic transducer
11b, 222, 322 Ultrasonic transmission member
12, 212, 312 Protruding portion
13, 213, 313 Tubular member
13a, 213a, 313a Groove
13b, 13c, 213b, 213c Through hole
14 Subject
14a Mucosa
14b Cell
15 Space
16 Introduction means
17 Extraction means
18 O-ring
221 Disc portion
223 Peripheral portion
224 Inclined portion
225 Protruding portion
Hmax Maximum dimension in longitudinal direction
NP Position corresponding to node
Wmax Maximum dimension in lateral direction

1. A mucosal cell collection device comprising:
an ultrasonic irradiation means;
a tubular member provided around the ultrasonic irradiation means and having a protruding portion protruding beyond one end of the ultrasonic irradiation means;
an introduction means configured to introduce liquid into a space formed by the ultrasonic irradiation means, the protruding portion of the tubular member, and a subject; and
an extraction means configured to extract a cell separated from mucosa of the subject from the space together with the liquid.

2. The mucosal cell collection device according to claim 1,
wherein the ultrasonic irradiation means includes an ultrasonic transducer and an ultrasonic transmission member configured to transmit ultrasound generated by the ultrasonic transducer.

3. The mucosal cell collection device according to claim 1,
wherein the introduction means and the extraction means each include a tube.

4. The mucosal cell collection device according to claim 1, comprising:
a sealing member between the ultrasonic irradiation means and the tubular member.

5. The mucosal cell collection device according to claim 2,
wherein the ultrasonic transducer includes a disc portion formed in a disc shape and is configured to be capable of vibrating in a lateral direction along a radial direction of the disc portion.

6. The mucosal cell collection device according to claim 5,
wherein the ultrasonic transmission member converts vibration of the ultrasonic transducer into vibration in a longitudinal direction orthogonal to the lateral direction.

7. The mucosal cell collection device according to claim 6,
wherein the ultrasonic transmission member is formed in a cymbal shape.

8. The mucosal cell collection device according to claim 7,
wherein the ultrasonic transmission member includes
a peripheral portion connected to the disc portion,
an inclined portion connected to the peripheral portion and extending at an angle away from the disc portion, and
a protruding portion connected to the inclined portion and protruding in a direction opposite to the disc portion.

9. The mucosal cell collection device according to claim 8,
wherein the tubular member is provided around the protruding portion.

10. The mucosal cell collection device according to claim 9,
wherein a maximum size in the lateral direction is 11 mm or less, and
a maximum size in the longitudinal direction is 10 mm or less.

11. The mucosal cell collection device according to claim 2,
wherein the ultrasonic transmission member is formed in a shape in which a cross-sectional area orthogonal to an axial direction of the ultrasonic transmission member decreases toward a tip of the protruding portion.

12. The mucosal cell collection device according to claim 11,
wherein the tubular member is formed in a shape in which a cross-sectional area orthogonal to an axial direction of the tubular member decreases toward the tip of the protruding portion.

13. The mucosal cell collection device according to claim 2,
wherein the tubular member is attached to the ultrasonic transmission member at a position corresponding to a node when the ultrasonic transmission member vibrates due to ultrasound generated by the ultrasonic transducer.

14. The mucosal cell collection device according to claim 13, comprising:
a sealing member between the ultrasonic transmission member and the tubular member,
wherein the sealing member is attached to the ultrasonic transmission member and/or the tubular member at the position corresponding to the node.

15. A method for collecting a mucosal cell using a mucosal cell collection device, the mucosal cell collection device including
an ultrasonic irradiation means,
a tubular member provided around the ultrasonic irradiation means and having a protruding portion protruding beyond one end of the ultrasonic irradiation means,
an introduction means configured to introduce liquid into a space formed by the ultrasonic irradiation means, the protruding portion of the tubular member, and a subject, and
an extraction means configured to extract a cell separated from mucosa of the subject from the space together with the liquid, the method comprising:
irradiating the mucosa of the subject with ultrasound through the liquid; and
extracting the cell separated from the mucosa of the subject together with the liquid.

16. The method for collecting a mucosal cell according to claim 15, further comprising:
washing away a substance in an oral cavity in the space and/or a substance in the oral cavity adhering to a surface of the mucosa of the subject with the liquid by filling the space formed by the ultrasonic irradiation means, the protruding portion of the tubular member, and the subject with the liquid and circulating the liquid prior to the irradiating or at an early stage of the irradiating.
